(19) United States
(12) Patent Application Publication
(10) Pub. No.: US 2013/0310840 A1
(43) Pub. Date: Nov. 21, 2013

(54) BONE CEMENT MIXING AND DELIVERY DEVICE AND METHOD OF USE

(71) Applicant: Cook Incorporated, (US)

(72) Inventor: Christopher G. Dixon, Bloomington, IN (US)

(21) Appl. No.: 13/673,020

(22) Filed: Nov. 9, 2012

Related U.S. Application Data
(63) Continuation of application No. 12/119,155, filed on May 12, 2008, now abandoned.

(51) Int. Cl. A61B 17/88

(52) U.S. Cl. CPC A61B 17/8833
USPC 606/94; 366/130

(57) ABSTRACT

In at least one embodiment of the present invention, a device for mixing and dispensing a bone cement mixture is provided. The device comprises a housing having a chamber configured to contain a first and second bone cement. A plunger including a plunger rod having a piston at a first end and a plunger handle at a second end is configured to actuate within the chamber. A mixing element is disposed adjacent the piston and is configured to rotate within the chamber to mix the first and second bone cement components to form the bone cement mixture. The plunger is configured to rotate the mixing element to mix the bone cement mixture. The plunger is also configured to advance through the chamber. As the plunger is advanced through the chamber, the piston is configured to receive the mixing element and dispense the bone cement mixture from the device.

### BACKGROUND OF THE INVENTION

1. Field of the Invention

The present invention relates to a device, a kit and a method for mixing and dispensing a bone cement mixture.

2. Description of Related Art

There is a clinical need to fill and stabilize damaged bones of patients, such as for example, filling defects in collapsed vertebra of patients suffering from severe back pain caused by osteoporosis, metastatic tumors or back injuries. Currently, these defects are repaired using multi-component bone cements that are mixed in open containers, transferred to a device and injected into the damaged bone where the mixture chemically reacts or cures to form a solid support structure.

The most widely used bone cements are based on polymethylmethacrylate (PMMA) and hydroxyapatite. These materials have relatively good strength characteristics, but have a number of drawbacks. These cements are a two-part chemically reactive system and have approximately five to ten minutes of working time once the components are mixed. As for example with the PMMA based system, one of the components is a liquid monomer methylmethacrylate (MMA), which is noxious and toxic to handle. The other component, the polymer component PMMA, is a powder that can be difficult to mix thoroughly. Moreover, current methods of mixing these two components together are typically done by hand in an open container or dish. This procedure permits significant vaporization of the noxious liquid monomer MMA. Also, the working time increases between mixing and dispensing because once the mixture is mixed it then needs to be transferred to a syringe for injection into the damaged bone. Moreover, the working time is limited because the viscosity of the cement constantly increases during mixing, thus making transferring of the mixture to the syringe and injection of the mixture into the damaged bone more difficult. Often, very high injection pressures and/or large bore needles may be necessary to inject the mixture, especially if it's near the end of the cements working time.

Thus, there is a need to provide a device for both mixing and dispensing a bone cement mixture.

### BRIEF SUMMARY OF THE INVENTION

Embodiments of the present invention provide a device, a kit and a method that facilitates mixing and dispensing of the bone cement mixture such that the interventionalist and the patient have minimal exposure to the noxious vapors of the monomer as well as providing more flexible working times for suitable injection-ability of the mixture into the damaged bone.

In at least one embodiment of the present invention, a device for mixing and dispensing a bone cement mixture is provided. The device comprises a housing having a longitudinal axis and a chamber formed within the housing for containing a first bone cement component. The housing has a distal end and a proximal end. An outlet at the distal end is in fluid communication with the chamber for receiving a second bone cement component and for dispensing the bone cement mixture from the device. A plunger is disposed cooperatively within the chamber and configured to actuate within the chamber, along the longitudinal axis. The plunger includes a plunger rod having a first end and a second end. A plunger rod includes a piston at the first end and a plunger handle at the second end configured to actuate the plunger within the chamber. A mixing element is disposed adjacent the piston. The plunger is configured to rotate the mixing element about the longitudinal axis to mix the first and second bone cement components together to form the bone cement mixture. The plunger is also configured to advance through the chamber from the proximal end toward the distal end. As the plunger advances through the chamber, the piston is configured to receive the mixing element and dispense the bone cement mixture from the chamber through the outlet.

In at least another embodiment of the present invention, a device for mixing and dispensing a bone cement mixture is provided. The device comprises a housing having a longitudinal axis and a chamber formed within the housing for containing a first bone cement component. The housing has a distal end and a proximal end. An outlet at the distal end is in fluid communication with the chamber for receiving a second bone cement component and for dispensing the bone cement mixture from the device. A plunger is disposed cooperatively within the chamber and configured to actuate within the chamber, along the longitudinal axis. The plunger includes a plunger rod having a first end and a second end. A plunger rod includes a piston at the first end and a plunger handle at the second end configured to actuate the plunger within the chamber. A mixing element is disposed adjacent the piston and configured to rotate about the longitudinal axis to mix the first and second bone cement components together to form the bone cement mixture. Turning the plunger handle in a first direction is configured to rotate the mixing element to mix the cement mixture. Turning the plunger handle in a second direction is configured to advance the plunger through the chamber from the proximal end toward the distal end. The piston is configured to receive the mixing element and dispense the bone cement mixture from the chamber through the outlet.

In at least another embodiment of the present invention, a bone cement substitute kit for mixing a bone cement mixture and dispensing the bone cement mixture into a damaged bone of a patient is provided. The kit comprises a first bone cement component and a second bone cement component. The kit further comprises a device including a housing having a longitudinal axis and a chamber formed within the housing for containing a first bone cement component. The housing has a distal end and a proximal end. An outlet at the distal end is in fluid communication with the chamber for receiving a second bone cement component and for dispensing the bone cement mixture from the device. A plunger is disposed cooperatively within the chamber and configured to actuate within the chamber. The plunger includes a plunger rod having a first end and a second end. The plunger rod includes a piston at the first end and a plunger handle at the second end configured to actuate the plunger within the chamber.

A mixing element is disposed adjacent the piston. The plunger is configured to rotate the mixing element about the longitudinal axis to mix the first and second bone cement components together to form the bone cement mixture. The plunger is also configured to advance through the chamber from the proximal end toward the distal end. As the plunger advances through the chamber, the piston is configured to receive the mixing element and dispense the bone cement mixture from the chamber through the outlet. The kit further comprises a needle in fluid communication with the outlet and configured to receive the bone cement mixture from the device and advance the cement mixture into the damaged bone of the patient.

In at least another embodiment of the present invention, a method for mixing a bone cement mixture and for dispensing the bone cement mixture into a damaged bone of a patient is provided. The method comprises providing a device including a housing having a longitudinal axis and a chamber formed therein. The housing has a distal end and a proximal end. An outlet at the distal end is in fluid communication with the chamber. A plunger is disposed cooperatively within the chamber and configured to actuate within the chamber. The plunger includes a plunger rod having a piston at a first end and a plunger handle at a second end. A mixing element is disposed adjacent the piston and configured to rotate about the longitudinal axis to mix the first and second bone cement components together to form the bone cement mixture.

The method further comprises introducing a first bone cement component and a second bone cement component into the chamber. A needle in fluid communication with the device is inserted into the damaged bone of the patient. The first bone cement component is mixed with the second bone cement component which includes actuating the plunger to rotate the mixing element about the longitudinal to mix the first and second bone cement components together to form the bone cement mixture. The bone cement mixture is dispensed into the damaged bone of the patient via the needle which includes actuating the plunger to advance through the chamber. As the plunger advances through the chamber, the piston receives the mixing element and dispenses the bone cement mixture from the chamber through the outlet. The bone cement is cured to stabilize the damaged bone of the patient.

Further objects, features and advantages of the invention will become apparent from consideration of the following description and the appended claims when taken in connection with the accompanying drawings.

### BRIEF DESCRIPTION OF THE DRAWINGS

FIG. 1 is a partial side view of a collapsed vertebra;

FIG. 2 is a partial side view of a device for stabilizing a collapsed vertebra in accordance with one embodiment of the present invention;

FIG. 3 is an enlarged view of FIG. 2;

FIG. 4 is a partial side view of a stabilized collapsed vertebra in accordance with one example of the present invention;

FIG. 5 is a partial side view of a device for stabilizing a collapsed vertebra in accordance with yet another embodiment of the present invention;

FIG. 6 is a partial side view of a device for stabilizing a collapsed vertebra in accordance with another embodiment of the present invention;

FIG. 7a is a side view of a device for stabilizing a collapsed vertebra in accordance with another embodiment of the present invention;

FIG. 7b is a perspective view of a device for stabilizing a collapsed vertebra in accordance with another embodiment of the present invention;

FIG. 7c is a side view of the device depicted in FIG. 7b;

FIG. 7d is a an end view of the device depicted in FIG. 7c taken along line 3;

FIG. 7e is a cross section of the device depicted in FIG. 7c taken along line 4;

FIG. 8a is a side view of a bone cement substitute kit in accordance with one embodiment of the present invention;

FIG. 8b is an exploded view of the bone cement substitute kit depicted in FIG. 8a; and

FIG. 9 is a flow chart for a method for stabilizing a damaged bone of a patient in accordance with an embodiment of the present invention.

### DETAILED DESCRIPTION OF THE INVENTION

Detailed embodiments of the present invention are disclosed herein. It is understood however, that the disclosed embodiments are merely exemplary of the invention and may be embodied in various and alternative forms. The figures are not necessarily to scale; some figures may be configured to show the details of a particular component. Therefore, specific structural and functional details disclosed herein are not to be interpreted as limiting but merely as a representative basis with the claims and for teaching one skilled in the art to practice of the present invention.

Examples of the present invention seek to overcome some of the concerns associated with stabilizing and/or repairing the damaged bone of a patient with a bone cement while minimizing toxic effects to both the patient and the interventionalist, and enhancing the ease of handling of the bone cement for both mixing and dispensing into the damaged bone.

Employing the principles of the present invention is, for example, a device, a kit, and a method for stabilizing and/or repairing a damaged bone of a patient. The device, which is utilized in both the kit and the method, is a closed mixing and dispensing system having a housing and a chamber formed therein for containing, for example, a first bone cement component. The housing includes an outlet in fluid communication with the chamber for receiving, for example, a second bone cement component. The device further includes a plunger disposed within the chamber configured to actuate within the chamber. The plunger includes a piston and a mixing element is disposed adjacent the piston. The mixing element is configured rotate within the housing to mix the first and second bone cement components together to form the bone cement mixture. The plunger is configured to actuate within the housing to advance the piston through the chamber.

As the piston advances through the chamber, the piston receives the mixing element and dispenses the bone cement mixture from the chamber through the outlet. The device may also be in fluid communication with a needle inserted into the damaged bone of a patient. The device is configured such that the bone cement mixture may be advanced from the device into the damaged bone of a patient via the needle. The device preferably mixes the bone cement components together without releasing noxious monomer fumes contained in one of the bone cement components. Moreover, since the device is configured to dispense the bone cement mixture there is no need for transferring the mixture from another source into the device. Accordingly, the device minimizes the mixing and dispensing time of the bone cement and thus, enhances the remaining working time for introducing the mixture into the damaged bone. Once the bone cement is introduced into the damaged bone of the patient it cures to form a solid structure which stabilizes the bone.

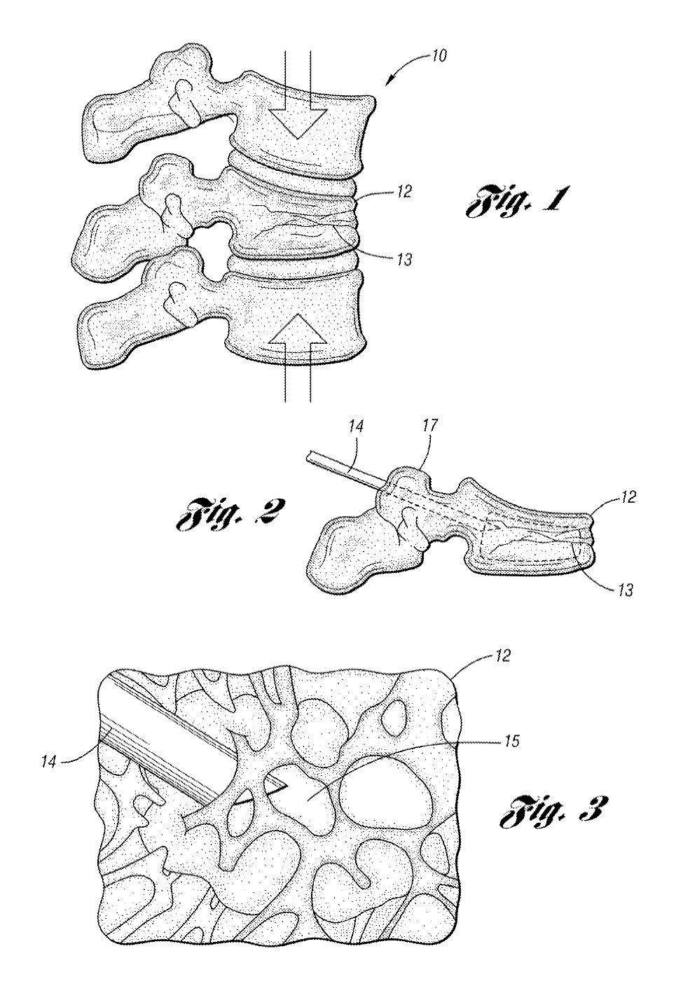

Referring now to the drawings, FIG. 1 illustrates a vertebra 10 which includes a collapsed vertebra 12 with a compression fracture 13. The vertebra 10 may be for example in the thoracic or lower spine of the patient. In the compression fracture 13 of the vertebra 12, the bone tissue of the vertebral body collapses. This condition is commonly caused by osteoporosis and less often by a tumor, or trauma to the back.

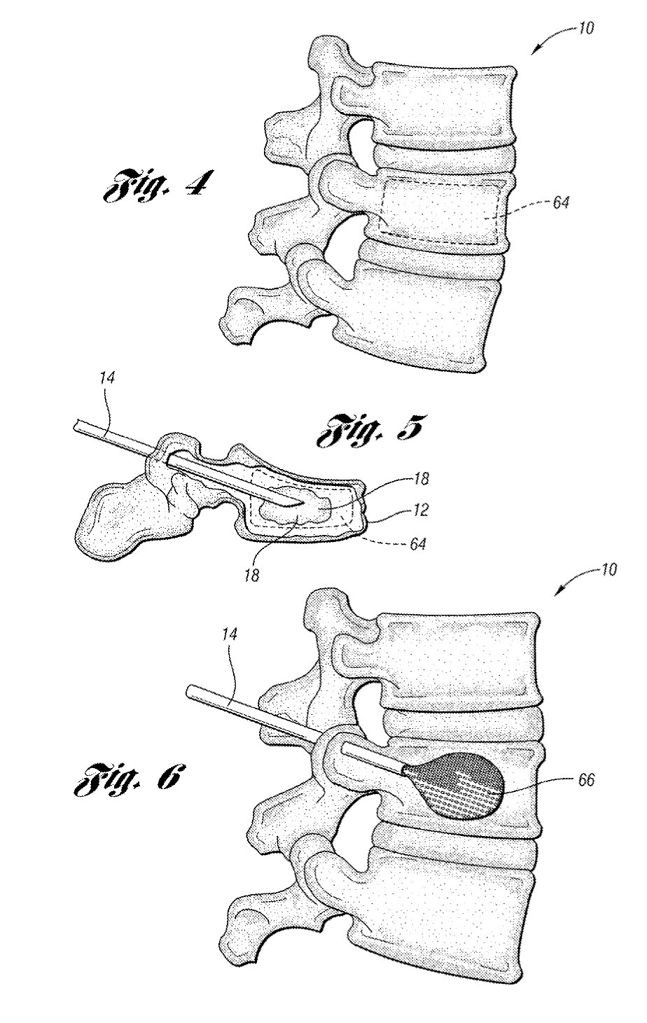

Referring now to FIGS. 2 and 3, at least one embodiment of the present invention is provided. The collapsed vertebra 12 may be stabilized by either verteborplasty or kyphoplasty, both of which are medical procedures for introducing bone cement into the collapsed vertebra. These procedures stabilize the collapsed vertebra by filling in open spaces within the vertebra 12 to provide a more continuous and solid form. Kyphoplasty may further stabilize the vertebra 12 by restoring vertebral spacing which alleviates nerve pinching from the vertebra 12. It should be noted that the present invention applies to both of these medical procedures and other procedures for stabilizing and/or repairing damaged bones of patients despite many of the various embodiments discussed herein are described using verteborplasty.

Vertebroplasty involves that a patient remain lying throughout the entire procedure. It is performed under a local anesthesia and/or a light sedative. A small nick is then made in the skin near the spine and a needle 14 is inserted percutaneously. As illustrated in FIG. 3, the needle 14 may be inserted into the interior open spaces 15 of the vertebra 12, for example via or through the left or right pedicle 17 of the vertebra 12.

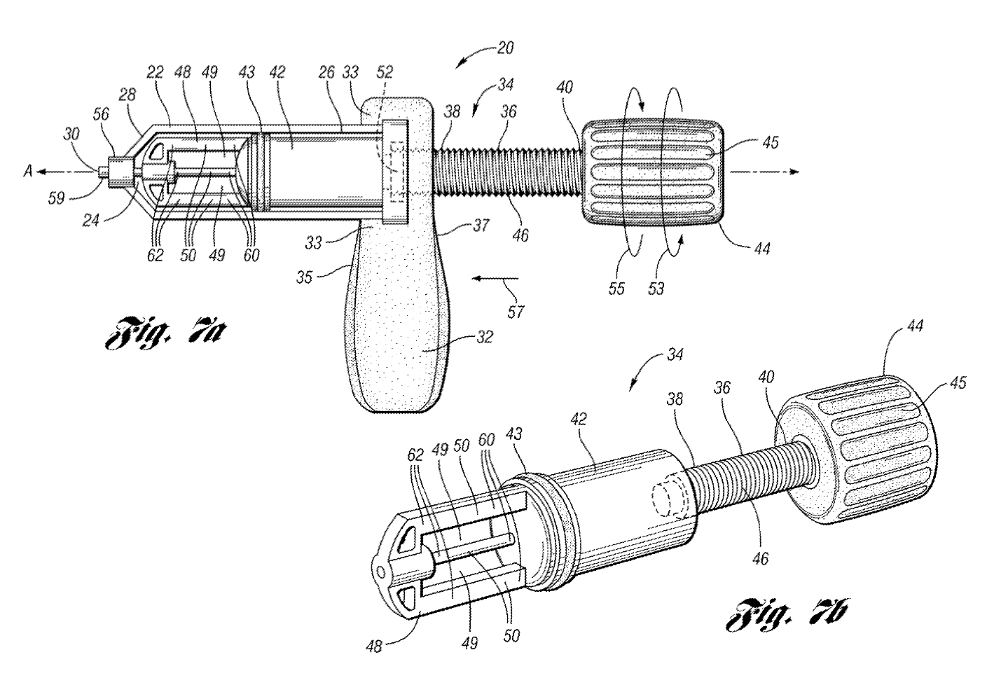

Referring to FIGS. 4 and 5, the bone cement mixture 18 may be dispensed from a device (not shown) through the needle 14 and into the vertebra 12 to form a solid structure 64 that supports the collapsed vertebra 12. The bone cement mixture 18 forms the solid structure 64 by chemically reacting or curing to become a solid. The stabilizing solid structure 64 may be formed within and/or about the collapsed vertebra 12 and may help restore vertebral spacing and alleviate nerve pinching by supporting the collapsed vertebra 12 generally in at least a compressive mode. Preferably, the structure substantially fills in the open spaces 15 of the collapsed vertebra 12 providing a more dense and continuous vertebra 12 which enhances mobility of the patient.

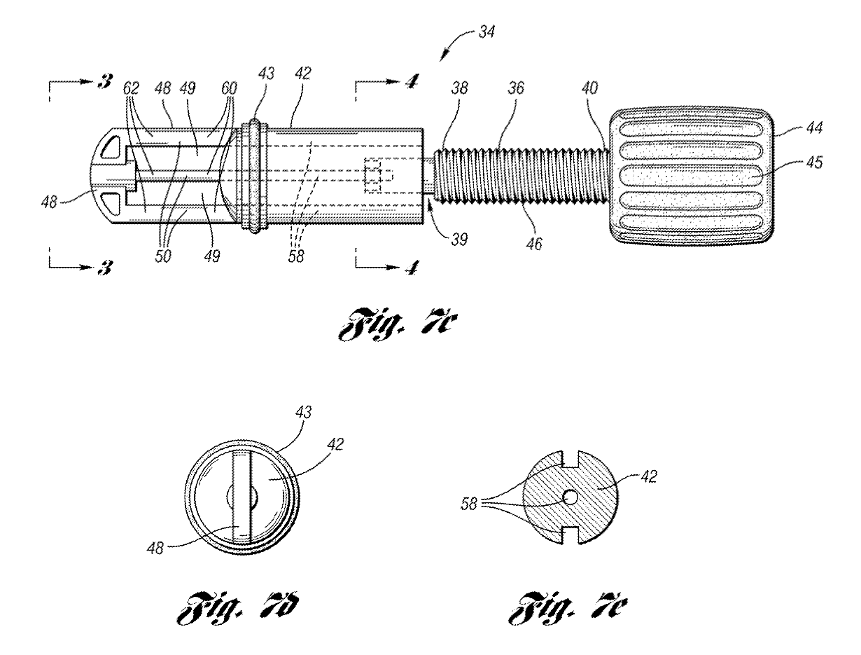

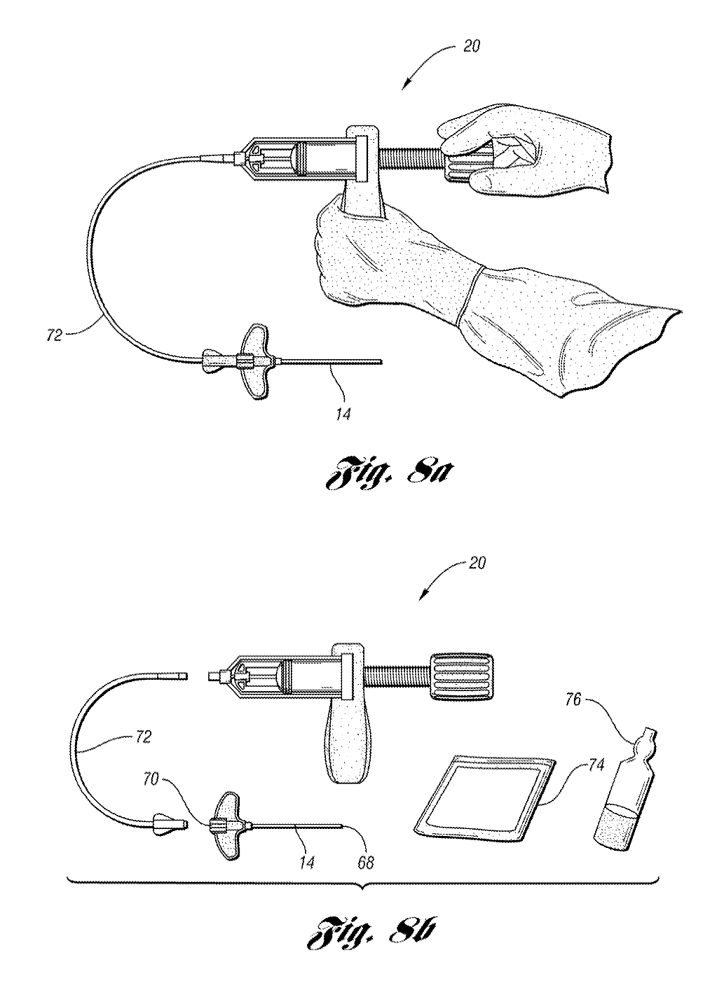

Referring to FIGS. 7a-c, at least one embodiment of a device for mixing a bone cement mixture and for dispensing the mixture is provided. In this embodiment, the device 20 or novel syringe comprises an injector housing 22 surrounding a chamber 24. The housing 22 has a substantially cylindrical shape disposed along a longitudinal axis A. The housing 22 has a proximal end 26 and a distal end 28 and includes an outlet 30 at the distal end 28 in fluid communication with the chamber 24. The chamber 24 is initially configured to contain a first bone cement component and is configured to receive a second bone cement component. For example, a bone cement system may be comprised of separate components A and B. These components may be a liquid and/or a solid, which when mixed together chemically react to form a solid structure. Component A may be initially contained in the chamber 24 and component B may be injected into the chamber 24 through the outlet 30 or vice versa.

To further illustrate, component A, for example, is a solid polymer powder of PMMA preloaded into the chamber 24 by the manufacturer of the device 20 and component B is a liquid monomer MMA with a relatively low viscosity of about 0.6 centipoise at room temperature. The MMA could be poured from an external container into the housing 22 prior to assembly of the device 20 or alternatively, injected into the outlet 30, via a syringe, through a luer fitting nozzle 56 at the distal end 28 of the housing 22. When the MMA is mixed with the PMMA, a chemically reacting paste may be formed which continually increases in viscosity over a working time of about 5 to 10 minutes to form a solid structure. In another example, component A may be sodium phosphate and component B may be solid powder of monocalcium phosphate, tricalcium phosphate, calcium carbonate or mixtures thereof that form a chemically reacting solution when mixed with sodium phosphate, which also increases in viscosity over a working time of about 5 to 10 minutes to form a solid structure.

In this embodiment, the device 20 further comprises a plunger 34 disposed within the chamber 24 and configured to actuate within the chamber 24. For example, the plunger 34 actuates by sliding within the chamber 24 towards the distal end 28 of the chamber 24. Preferably, the plunger 34 is a screw-gear plunger. The screw-gear plunger 34 includes a plunger rod 36 having a distal end 38 and a proximal end 40. In this embodiment, the screw-gear plunger 34 includes a piston 42 disposed at the distal end 38 of the plunger rod 36 and a plunger handle 44 at the proximal end 40 of the plunger rod 36. The piston 42 includes a seal member 43 between the piston 42 and the chamber 24 for preventing bone cement from flowing therebetween and to facilitate movement of the piston 42 within the chamber 24. Preferably, the seal member 43 comprises an o-ring. The plunger rod 36 includes male threads or grooves 46 formed thereon.

In this embodiment, a mixing element 48 is disposed adjacent the piston 42 and configured to rotate about the longitudinal axis A to mix the first and second bone cement components together within the chamber 24 to form the bone cement mixture 18. The mixing element 48 includes a structure to promote mixing of the bone cement components, such as a mixing paddle having a plurality arms 50 defining a plurality of apertures 49. As illustrated in FIGS. 7c and 7e, the piston 42 is configured to rotate about the longitudinal axis A and has a plurality of slots 58 formed therein which correspond with the arms 50. Each of the arms 50 has a first portion 60 and a second portion 62 extending beyond the first portion 60. The first portions 60 are disposed within the corresponding slots 58 to engage the mixing paddle 48 with the piston 42 such that mixing paddle 48 and the piston 42 rotate cooperatively about the longitudinal axis A during mixing of the bone cement components. In a preferred embodiment, the arms 50 and the slots 58 form a press-fit seal to prevent seepage of the bone cement through the channels defined by the slots 58. During dispensing of the bone cement mixture 18 from the device 20, the slots 58 receive the second portions 62 of the arms 50 of the mixing paddle 48 as the piston 42 advances through the chamber 24 from the proximal end 26 towards the distal end 28.

In a preferred embodiment, the chamber 24, which is configured to receive the screw-gear plunger 34 at its proximal end 26, is a high pressure injection chamber. The high pressure injection chamber 24 is adapted for withstanding positive displacement pressures associated with advancing “paste like” fluids through the outlet 30. In one example, the viscosity of the “paste like” fluid is greater than about 1,000 centipoise. The high pressure chamber 24 is preferably made of a plastic, such as polycarbonate, but may be made of glass or other suitable materials known in the art.

In this embodiment, the device 20 includes an injector handle 32 for gripping the device 20 by an interventionalist. The injector handle 32 supports the housing 22, and thus the high pressure chamber 24, at its proximal end 26. As shown in FIG. 7a, the first end 35 of the injector handle 32 includes flanges or tabs 33 configured to receive the proximal end 26 of the housing 22. The injector handle 32 includes an opening 52 through which the screw-gear plunger 34 enters the high pressure chamber 24 from the opposing second end 37 of the injector handle 32. The piston 42 and the mixing element 48 may initially be detached from the plunger rod 36 before the plunger rod 36 enters the opening 52 at the second end 37. The piston 42 and the mixing element 48 may be reattached to the distal end 38 of the plunger rod 36 after the plunger rod 36 passes through the opening 52 of the injector handle 32. As the housing 22 is received by the tabs 33 at the first end 35 of the injector handle 32, the piston 42 and the mixing element 48 slide within the high pressure chamber 24.

In this embodiment, the screw-gear plunger 34 is used to mix the bone cement components to form the bone cement mixture as well as dispense the bone cement mixture from the device 20. In this embodiment, the screw-gear plunger 34 enters the opening 52 formed within the injector handle 32. Preferably, the grooves 46 of the plunger rod 36 do not extend along the entire length of the plunger rod 36. For example, the distal end 38 of the plunger rod 36 includes a non-grooved portion 39 which does not have any grooves disposed thereon. In this embodiment, the opening 52 is a threaded opening having female threads configured to engage/disengage the grooves 46 of the plunger rod 36 and thereby adjust the device 20 between a disengaged position and an engaged position.

In this embodiment, the threaded opening 52 initially receives the non-grooved portion 39 of the plunger rod 36, defining the disengaged position. In the disengaged position, an interventionalist turns the plunger handle 44 to actuate the screw-gear plunger 34 to rotate the mixing element 46 about the longitudinal axis A within the chamber to mix the bone cement components together. The process of rotating the mixing element 48 to mix the bone cement mixture within the high pressure injection chamber 24 may be repeated until the bone cement components are thoroughly mixed together to the satisfaction of the interventionalist.

In a preferred embodiment, the device 20 is adjusted to the engaged position by pushing or nudging the screw-gear plunger 34 forward, in the direction of arrow 57, so that the threaded opening 52 engages the grooves 46 of the plunger 36. In the engaged position, the interventionalist turns the plunger handle 44 and the threaded opening 52 cooperates with the grooves 46 to advance the screw-gear plunger 34, and thus the piston 42, through the high pressure injection chamber 24 towards the distal end 28 of the housing 22. As the screw-gear plunger 34 advances towards the distal end 28 of the housing 22, the piston 42 receives the mixing element 48 and advances the bone cement mixture from the high pressure injection chamber 24 through the outlet 30, thereby dispensing the mixture from the device 20. Simultaneously, turning the plunger handle 44 when the device 20 is in the engaged position may also rotate the mixing element 48 as the plunger 34 is advanced.

In a preferred embodiment, turning the plunger handle 44 in a first direction, for example a counter-clockwise direction 53, when the device 20 is in the disengaged position, actuates the screw-gear plunger 34 to rotate the mixing element 46 about the longitudinal axis A within the chamber 24 to mix the bone cement components together. Whereas, turning the plunger handle 44 in a second direction, for example a clockwise direction 55, when the device 20 is in the engaged position, actuates the screw-gear plunger 34 to advance through the chamber 24. In this embodiment, the threaded opening 52 and the grooved portion of the plunger rod 36 may be configured to engage and cooperate to advance the screw-gear plunger 34 through the chamber 24 only when the plunger handle 44 is turned in the second direction. This acts as a safety mechanism to prevent inadvertent advancement of the screw-gear plunger 34 through the chamber when the intervertionalist turns the plunger handle 44 in the first direction to mix the bone cement mixture. Thus, the interventionalist is thereby prevented from prematurely dispensing an insufficiently mixed bone cement mixture.

The foregoing embodiments have been given solely by way of example. It will be understood by those skilled in the art that various modifications may be made without departing from the spirit and scope of the invention. For example, the device 20 may include any suitable engagement/disengagement mechanism which allows rotation of the mixing element 48 (i.e., mixing of the bone cement mixture) when the plunger handle 44 is turned in one direction and allows advancement of the screw-gear plunger 34 (i.e., dispensing of the bone cement mixture) when the plunger handle 44 is turned in another direction.

The device 20 may further include as part of the outlet 30, a luer fitting nozzle 56. The bone cement mixture may be dispensed from the outlet 30 of the device 20 through the luer fitting nozzle 56. In this example, the luer fitting nozzle 56 typically has a tapered end 59 that facilitates connecting to various types of cannula, tubing, needles or other similar medical devices.

In at least one embodiment, the plunger handle 44 of the screw-gear plunger 34 is positioned along the plunger rod 36 and disposed outside of the high pressure chamber 24 and has, for example, a cylindrical shape and a plurality of gripping indents 45 for facilitating gripping and turning of the screw-gear plunger 34 by the interventionalist.

Referring to FIG. 6, at least one other embodiment for stabilizing a collapsed vertebra 12 of a patient is provided. A method for dispensing the bone cement mixture into a damaged bone of a patient may include placing a balloon 66 into the collapsed vertebra 12. The balloon 66 may be positioned in the vertebra 12 for example via the needle 14, a catheter or mandrel. The balloon 66 is then filled with the bone cement mixture and sealed. The balloon 66 may be sealed for example by twisting the needle 14 and shearing the corresponding end portion of the balloon 66 or alternatively by applying any suitable adhesive, such as a cyanoacrylate, to the end portion. The cement mixture within the sealed balloon 66 cures to form a solid support structure 64 within the collapsed vertebra 12.

In this embodiment, the balloon 66 may be made of any suitable material used for medical intracorporeal balloon devices. However, a polymer impermeable to body fluids and MMA may be preferred. An example of such material is polyethylene terephthalate (PET) or polybutylene terephthalate (PBT).

The interventionalist may also assess whether the collapsed vertebra 12 is sufficiently filled via fluoroscopy. If the collapsed vertebra 12 is not sufficiently filled, an additional balloon may be placed within the collapsed vertebra 12 and the filling, solidifying and/or curing and sealing steps may be repeated.

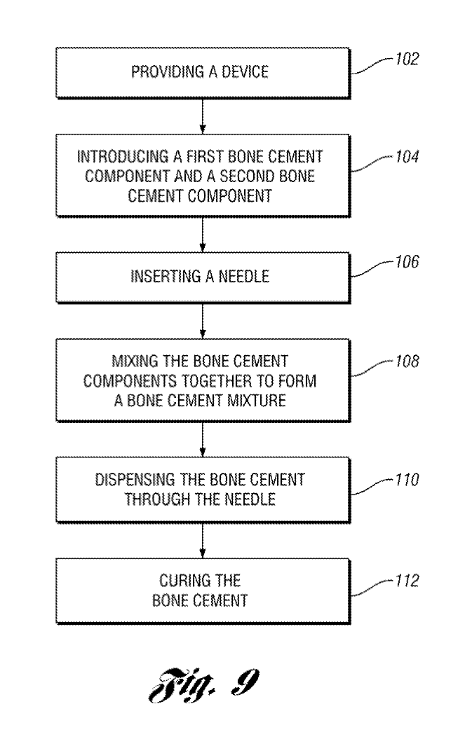

Referring also to FIGS. 8a-b, at least one embodiment of a bone cement substitute kit is provided. The kit includes a device 20 as discussed in the forgoing paragraphs as well as the needle 14 which is configured for fluid communication with the device 20 and for advancing the bone cement mixture into the collapsed vertebra 12. The needle 14 may have a beveled edge end 68 for easy insertion and removal from the collapsed vertebra 12. The other end 70 of the needle 14 may be directly coupled to the device 20 or indirectly coupled via tubing 72. The tubing 72 provides fluid communication between the device 20 and the needle 14. Preferably, the tubing 72 may be flexible to facilitate maneuvering of the device 20 during injection of the bone cement into the damaged bone.

The kit may further comprise a sealed envelope 74 containing a component of the bone cement, such as PMMA, and sealed container 76 containing the other component of the bone cement, such as MMA. Alternatively, either of the first and second components of the bone cement may already be contained within the chamber 24 of the device 20 as packaged. The device 20 may include a valve which closes off fluid communication between the chamber 24 and the outlet 30 to facilitate packaging and handling of the kit when a bone cement component is pre-packaged within the chamber 24. Additionally, the kit may further include a balloon 66 (shown in FIG. 6) for receiving the bone cement mixture 18.

Referring to FIG. 9, a method for mixing a bone cement mixture and dispensing the bone cement mixture into a damaged bone of a patient is provided. The method includes providing a device (102) as discussed in the forgoing paragraphs.

A first bone cement component and a second bone cement component are introduced (104) into a chamber.

A needle is inserted (106) into the damaged bone of the patient. The needle is in fluid communication with the apparatus.

The first and second bone cement components are mixed together (108) to form a bone cement mixture. This includes actuating a plunger to rotate a mixing element about a longitudinal axis to mix the bone cement components. Actuating the plunger to rotate the mixing element may include positioning a plunger rod in a disengaged position and turning a plunger handle in a first direction.

The bone cement mixture is dispensed from the device into the damaged bone of the patient via the needle (110). This includes actuating the plunger to advance a piston through the chamber of the device, where the piston receives the mixing element and advances the bone cement mixture from the chamber and through the needle. Actuating the plunger to advance a piston through the chamber of the device may include positioning a plunger rod in an engaged position and turning the plunger handle in a second direction. The bone cement mixture is then allowed to cure (112) to stabilize the damaged bone of a patient.

The method may further comprise positioning a balloon within the damaged bone of the patient, wherein dispensing the bone cement mixture includes receiving the bone cement mixture into the balloon via the needle.

As a person skilled in the art will readily appreciate, the above description is meant as an illustration of the implementation of the principles of this invention. This description is not intended to limit the scope or application of this invention in that the invention is susceptible to modification variation and change, without departing from the spirit of this invention, as defined in the following claims.

1. A device for mixing and dispensing a bone cement mixture, the device comprising:
a housing having a longitudinal axis and a chamber formed therein for containing a first bone cement component, the housing having a distal end and a proximal end, the housing including an outlet at the distal end in fluid communication with the chamber for receiving a second bone cement component and for dispensing the bone cement mixture from the device;
a plunger disposed cooperatively within the chamber and configured to actuate within the chamber, the plunger including a plunger rod having a first end and a second end, the plunger rod having a piston at the first end and a plunger handle at the second end configured to actuate the plunger within the chamber; and
a mixing element disposed adjacent the piston, the plunger configured to rotate the mixing element about the longitudinal axis to mix the first and second bone cement components together to form the bone cement mixture, the plunger configured to advance through the chamber from the proximal end toward the distal end, wherein the piston is configured to receive the mixing element and dispense the bone cement mixture from the chamber through the outlet as the plunger advances through the chamber.

2. The device of claim 1 further comprising a handle having a first end, a second end, and a threaded opening formed therethrough, the handle being configured to support the housing at the first end, the handle being configured to receive the plunger rod through the threaded opening at the second end, the plunger rod including a first portion having grooves disposed thereon and a second portion having no grooves.

3. The device of claim 2 wherein the threaded opening cooperates with the second portion of the plunger rod to define a disengaged position, wherein the threaded opening cooperates with the first portion of the plunger rod to define an engaged position in which the threaded opening engages the grooves of the first portion.

4. The device of claim 3 wherein turning the plunger handle when the plunger rod is in the disengaged position is configured to rotate the mixing element to mix the first and second bone cement components together.

5. The device of claim 3 wherein turning the plunger handle when the plunger rod is in the engaged position is configured to advance the plunger through the chamber, wherein the piston is configured to receive the mixing element and dispense the bone cement mixture.

6. The device of claim 3 wherein turning the plunger handle in a first direction when the plunger rod is in the disengaged position is configured to rotate the mixing element to mix the first and second bone cement components together.

7. The device of claim 6 wherein turning the plunger handle in a second direction when the plunger rod is in the engaged position is configured to advance the plunger through the chamber, wherein the piston is configured to receive the mixing element and dispense the bone cement mixture.

8. The device of claim 5 wherein turning the plunger handle when the plunger rod is in the engaged position is configured to rotate the mixing element and advance the plunger through the chamber.

9. The device of claim 1 wherein turning the plunger handle in a first direction is configured to rotate the mixing element to mix the bone cement mixture, wherein turning the plunger handle in a second direction is configured to advance the plunger through the chamber.

10. The device of claim 1 wherein the mixing element includes a plurality of arms defining a plurality of apertures, each of the arms having a first portion and a second portion extending beyond the first portion, wherein the piston includes a plurality of slots which correspond with the plurality of arms, wherein the first portion of each of the arms is configured to cooperate with each of the slots to engage the mixing element with the piston such that the mixing element and the piston are configured to rotate cooperatively about the longitudinal axis when the bone cement components are mixed, wherein the slots are configured to receive the second portion of the arms when the plunger is advanced through the chamber.

11. A bone cement substitute kit for mixing a bone cement mixture and dispensing the bone cement mixture into a damaged bone of a patient, the kit comprising:
a first bone cement component and a second bone cement component;
a device including:
a housing having a longitudinal axis and a chamber formed therein for containing a first bone cement component, the housing having a distal end and a proximal end, the housing including an outlet at the distal end in fluid communication with the chamber for receiving a second bone cement component and for dispensing the bone cement mixture from the device;
a plunger disposed cooperatively within the chamber and configured to actuate within the chamber, within the chamber, the plunger including a plunger rod having a first end and a second end, the plunger rod having a piston at the first end and a plunger handle at the second end configured to actuate the plunger within the chamber; and
a mixing element disposed adjacent the piston, the plunger configured to rotate the mixing element about the longitudinal axis to mix the first and second bone cement components together to form the bone cement mixture, the plunger configured to advance through the chamber from the proximal end toward the distal end, wherein the piston is configured to receive the mixing element and dispense the bone cement mixture from the chamber through the outlet as the plunger advances through the chamber; and
a needle in fluid communication with the outlet and configured for receiving the bone cement mixture from the device and for advancing the bone cement mixture into the damaged bone of the patient.

12. The kit according to claim 11 further comprising a handle having a first end, a second end, and a threaded opening formed therethrough, the handle being configured to support the housing at the first end, the handle being configured to receive the plunger rod through the threaded opening at the second end, the plunger rod including a first portion having grooves disposed thereon and a second portion having no grooves, wherein the threaded opening cooperates with the second portion of the plunger rod to define a disengaged position, wherein turning the plunger handle when the plunger rod is in the disengaged position is configured to rotate the mixing element to mix the first and second bone cement components together, wherein the threaded opening cooperates with the first portion of the plunger rod to define an engaged position in which the threaded opening engages the grooves of the first portion, wherein turning the plunger handle when the plunger rod is in the engaged position is configured to advance the plunger through the chamber, wherein the piston is configured to receive the mixing element and dispense the bone cement mixture.

13. The kit according to claim 11 wherein turning the plunger handle in a first direction is configured to rotate the mixing element to mix the bone cement mixture, wherein turning the plunger handle in a second direction is configured to advance the plunger through the chamber.

14. A method for mixing a bone cement mixture and dispensing the bone cement mixture into a damaged bone of a patient, the method comprising:
providing a device including a housing having a longitudinal axis and a chamber formed therein, the housing having a distal end and a proximal end, an outlet at the distal end in fluid communication with the chamber, a plunger disposed cooperatively within the chamber and configured to actuate within the chamber, the plunger including a plunger rod having a piston at a first end and a plunger handle at a second end, a mixing element disposed adjacent the piston and being configured to rotate about the longitudinal axis to mix the first and second bone cement components together to form the bone cement mixture, the piston being configured to receive the mixing element;
introducing a first bone cement component and a second bone cement component into the chamber;
inserting a needle into the damaged bone of the patient, the needle being in fluid communication with the device;
mixing the first bone cement component with the second bone cement component within the chamber to form the bone cement mixture;
dispensing the bone cement mixture from the device into the damaged bone of the patient via the needle; and
curing the bone cement mixture to stabilize the damaged bone of the patient.

15. The method of claim 14 wherein the device further includes a handle having a first end, a second end, and a threaded opening formed therethrough, the handle being configured to support the housing at the first end, the handle being configured to receive the plunger rod through the threaded opening at the second end, the plunger rod including a first portion having grooves disposed thereon and a second portion having no grooves, wherein the threaded opening cooperates with the second portion of the plunger rod to define a disengaged position, wherein the threaded opening cooperates with the first portion of the plunger rod to define an engaged position in which the threaded opening engages the grooves of the first portion.

16. The method of claim 15 wherein the step of mixing the first bone cement component with the second bone cement component includes actuating the plunger to rotate the mixing element about the longitudinal axis, wherein the step of dispensing the bone cement mixture from the device includes actuating the plunger to advance through the chamber, wherein the piston is configured to receive the mixing element and dispense the bone cement mixture from the chamber through the outlet and into the needle.

17. The method according to claim 18 wherein the step of actuating the plunger to rotate the mixing element includes positioning the plunger rod in the disengaged position and turning the plunger handle, wherein the step of actuating the plunger to advance the piston through the chamber includes positioning the plunger rod in the engaged position and turning the plunger handle.

18. The method according to claim 19 wherein turning the plunger handle in a first direction is configured to rotate the mixing element, wherein turning the plunger handle in a second direction is configured to advance the plunger.

19. The method according to claim 18 wherein turning the plunger handle in the second direction when the plunger rod is in the engaged position is configured to rotate the mixing element as the plunger advances through the chamber.

20. The method of claim 14 further comprising positioning a balloon within the damaged bone of the patient, wherein the step of dispensing the bone cement mixture includes receiving the bone cement mixture into the balloon via the needle.
